(19) United States
(12) Patent Application Publication
(10) Pub. No.: US 2008/0096275 A1
(43) Pub. Date: Apr. 24, 2008

(54) Protein For Improving Cell-Attachment Efficiency and Use Thereof

(75) Inventors: Chia-Hui Lin, Taichung City (TW); Tzu-Wei Wang, Taipei County (TW)

Correspondence Address: WPAT, PC;INTELLECTUAL PROPERTY ATTORNEYS, 2030 MAIN STREET, SUITE 1300, IRVINE, CA, 92614, US

(73) Assignee: BIO999 INC., Yunlin County (TW)

(21) Appl. No.: 11/552,507

(22) Filed: Oct. 24, 2006

(51) Int. Cl. C07K 14/435; C12N 5/02

(52) U.S. Cl. 435/402; 530/350

(57) ABSTRACT

The present invention provides a method for improving cell-attachment efficiency comprising (a) preparing a protein in aqueous solution, wherein the protein has a formula A-B-C, wherein A represents a GRGDS amino acid sequence; B represents a cellulose binding domain (CBD); and C represents a GRGDS amino acid sequence, an RGD amino acid sequence, or an amino acid sequence of growth factor; (b) coating the protein solution into a carrier; and (c) seeding cells onto the carrier.

### FIELD OF THE INVENTION

The present invention relates to a method for improving cell-attachment efficiency. The present invention further relates to a protein for improving cell-attachment efficiency.

### BACKGROUND OF THE INVENTION

The proper function of the tissue-engineering scaffold is to guide cells' infiltration and segregation, which eventually helps cells develop into a real tissue in vitro (Urech L, et al., Biomaterials 2005; 26: 1369-1379; Burdick J A, et al., J Biomed Mater Res 2002; 63: 484-491). Scaffold modification provides an approach to control cell migration and promote tissue ingrowth (Ikada Y, et al., Biomaterials 1994; 15: 725-736). Most of the modification methods are to immobilize adhesion molecules onto the surface of tissue-engineering scaffold. These molecules are common substances existing in the extracellular matrix, such as fibronectin, vitronectin, or laminin. These proteins then regulate the adhesion, migration, and growth of cells by binding to integrin receptors, located on the outer cellular membranes (Plow E F, et al., J Biol Chem 2000; 275: 21785-21788; van der Flier A, et al., Cell Tissue Res 2001; 305: 285-298).

Capable of cell attachment, differentiation, and migration, fibronectin is a ubiquitous glycoprotein macromolecule that can be found in the extracellular matrix of all vertebrates (Stidwill R P, et al., Cell Motil Cytoskeleton 1998; 41: 68-73). Arg—Gly—Asp (RGD) was found to be a major functional amino acid sequence responsible for cellular adhesion (Pierschbacher M D et al., Nature 1984; 309(5963): 30-33). The RGD sequence on the fibronectin can be recognized by the integrin receptor, so as to rearrange the internal structure of the cell, and enable the cells to attach onto the extracellular matrix steadily (Hynes R O, Cell 1992; 69: 11-25). If immobilized on the scaffold, RGD sequence also improved cell adhesion as fibronectin does. However, the immobilization of RGD or fibronectin generally needs tedious crosslinking process with very low immobilization yield, and is easily denatured or lost in 3-D conformation because of the adhesion molecule reaction sites being buried inside the scaffold or consumed for immobilization (Hersel U, et al., Biomaterials 2003; 24: 4385-4415; Ugarova T P, et al., Biochemistry 1995; 34: 4457-4466). In addition, the immobilization is always carried out in nonaqueous solutions, which cause the adhesion molecule to lose its biological activity. It is also mentioned that the whole immobilization process is time-inefficient, cost-expensive, and can possibly activate platelets adhesion after implantation (Chiba M, et al., J Immunol Methods 1996; 191: 55-63).

U.S. Pat. No. 6407,208 provides a chimeric protein with a cellulose binding domain. U.S. Application No. 2004/0005690 provides a modified CBD/RGD recombinant attachment factor for improving cell-attachment efficiency and the manufacturing method thereof.

### BRIEF DESCRIPTION OF THE DRAWINGS

FIG. 1 shows MTT assay of P—BFP and N—BFP group for (a) DFs and (b) keratinocytes attached at the first 72 h. In this figure, N—BFP means the group without BFP coated on petri dish, and P—BFP means the group with BFP coated on petri dish.

FIG. 2 shows Total DNA analysis of P—BFP and N—BFP group for (a) DFs and (b) keratinocytes attached and cultured for different time periods. In this figure, N—BFP represents the group without BFP coated on petri dish, and P—BFP represents the group with BFP coated on petri dish.

FIG. 3 shows OD value of LDH in the medium to evaluate the cytotoxic effects of BFP in different concentrations on (a) DFs, and (b) keratinocytes. In this figure, BFP represents bifunctional RGD-containing fusion protein, SFM represents serum-free medium, and DF represents DMEM+10% FBS.

FIG. 4 shows Keratinocytes adhesion (a) without BFP coated substrate and (b) with BFP coated substrate were observed under SEM, 2 h after seeding. Whereas (c) and (d) are general SEM examination of keratinocytes adhesion of 8 h after seeding without BFP coated substrate and with BFP coated substrate, respectively.

FIG. 5(a) shows the morphology of keratinocyte examined under inverted microscope on the third day after seeding. (b) shows keratinocytes cultured on BFP coated petri dish for 14 days and stained with anti-mouse IgG HRP secondary antibody applied, but without monoclonal anti-pan cytokeratin primary antibody conjugated as negative control. (c) shows keratinocytes cultured on BFP coated petri dish for 14 days and treated with monoclonal anti-pan cytokeratin with first and second antibody applied. In this FIG. 5(c), arrowhead denotes dark-brown stain.

FIG. 6 shows the morphological comparison and cell adhesion numbers of keratinocytes (a,b,c) and DFs (d,e,f). Keratinocytes culture for 3 days on (a) BFP uncoated plate (b) RGD graft plate (c) BFP coated plate. DFs culture for 3 days on (d) BFP uncoated plate (e) RGD graft plate and (f) BFP coated plate.

### SUMMARY OF THE INVENTION

The present invention provides a method for improving cell-attachment efficiency comprising (a) preparing a protein in aqueous solution, wherein the protein has a formula A-B-C, wherein A represents a GRGDS amino acid sequence; B represents a cellulose binding domain (CBD); and C represents a GRGDS amino acid sequence, an RGD amino acid sequence, or an amino acid sequence of growth factor; (b) coating the protein solution into a carrier; and (c) seeding cells onto the carrier.

The present invention also provides a protein for improving cell-attachment efficiency which has a formula A-B-C

wherein

A represents a GRGDS amino acid sequence;

B represents a cellulose binding domain (CBD); and

C represents a GRGDS amino acid sequence, an RGD amino acid sequence, or an amino acid sequence of growth factor.

### DETAILED DESCRIPTION OF THE INVENTION

### Term Definition

The term “cell adhesion molecules (CAMs)” refers to proteins located on the cell surface involved with the binding with other cells or with the extracellular matrix (ECM) in the process called cell adhesion.

The term “tissue engineering” refers to the use of a combination of cells, engineering materials, and suitable biochemical factors to improve or replace biological functions in an effort to affect the advancement of medicine.

The term “tissue engineering scaffold” refers to an artificial structure capable of supporting three-dimensional tissue formation. These scaffolds are often critical, both in vitro as well as in vivo, to recapitulating the in vivo milieu and allowing cells to influence their own microenvironments. Such devices, usually referred to as scaffolds, serve at least one of the following purposes: Allow cell attachment and migration, deliver and retain cells and biochemical factors, enable diffusion of vital cell nutrients and expressed products, and exert certain mechanical and biological influences to modify the behavior of the cell phase.

The term “growth factor” refers to a naturally occuring protein capable of stimulating cellular proliferation and cellular differentiation. Growth factors are important for regulating a variety of cellular processes.

The term “bifunctional fusion protein (BFP)” refers to a recombinant protein in which the N-terminal and the C-terminal sequence can simultaneously serve two particular biological functions.

The present invention provides a method for improving cell-attachment efficiency comprising (a) preparing a protein in aqueous solution, wherein the protein has a formula A-B-C, wherein A represents a GRGDS amino acid sequence; B represents a cellulose binding domain (CBD); and C represents a GRGDS amino acid sequence, an RGD amino acid sequence, or an amino acid sequence of growth factor; (b) coating the protein solution into a carrier; and (c) seeding cells onto the carrier.

In the preferred embodiment, the CBD is isolated from Trichoderma koningii cellobiohydrolase I gene (CBH I gene). The CBH I gene(68 kDa) comprises a C-terminal CBD (37 amino acids) connected to an N-terminal catalytic domain by a proline and threonine-rich linker (PT linker) in the formula A-B-C. The PT linker is positioned between A and B or B and C.

In the preferred embodiment, the GRGDS sequence expresses in a stable annular structure or in a linear-free form. Moreover, the annular structure is built by disulfide bonds of cysteine residues.

The method of the present invention further comprises a step of shaking the carrier for fixation of the protein between step (b) and step (c). In the preferred embodiment, the method further comprises a step of drying the carrier to remove residual liquid between shaking step and step (c). Furthermore, the method is carried out in aqueous solution in the absence of any organic solvent or crosslinking agent without losing the 3D conformation of bio-molecules.

The cell type used by the method of the present invention is not limited but to include any live cell such as alive cell, skin cell, stem cell or other delayed-action attachment cell. In the preferred embodiment, the skin cell is human epidermal keratinocyte or dermal fibroblast.

In the method of present invention, MTT assay, total DNA measurement, and lactate dehydrogenase analysis are used to evaluate the cell viability, cell proliferation, and cytotoxicity. The immunochemical stain and SEM examination are chosen to make sure that the cultured cells still kept in phenotype. The present invention proves that the BFP is successfully coated on the petri dish to improve the cells attachment efficiency. The whole coating procedure is just done in aqueous solution without any organic solvent being involved. The method of present invention is much simpler than the traditional one, and there is no possibility to damage the immobilized bio-molecules. In the preferred embodiment, BFP enhances attachment of keratinocytes and dermal fibroblasts without losing normal cell morphology and keep keratinocytes on the desired differentiation pathway.

The method of present invention could be applied to cosmetics for improving skin health.

The present invention also provides a protein for improving cell-attachment efficiency which has a formula A-B-C, wherein A represents a GRGDS amino acid sequence; B represents a cellulose binding domain (CBD); and C represents a GRGDS amino acid sequence, an RGD amino acid sequence, or an amino acid sequence of growth factor.

In the preferred embodiment, the CBD is isolated from Trichoderma koningii cellobiohydrolase I gene (CBH I gene). The CBH I gene(68 kDa) comprises a C-terminal CBD (37 amino acids) connected to an N-terminal catalytic domain by a proline and threonine-rich linker (PT linker) in the formula A-B-C. The PT linker is positioned between A and B or B and C.

In the preferred embodiment, the GRGDS sequence expresses in a stable annular structure or in a linear-free form. Moreover, the annular structure is built by disulfide bonds of cysteine residues.

The protein of present invention could be applied to cosmetics for improving skin health.

### EXAMPLES

The following examples are offered by way of illustration and not by way of limitation.

### Example 1

### Preparation of BFP

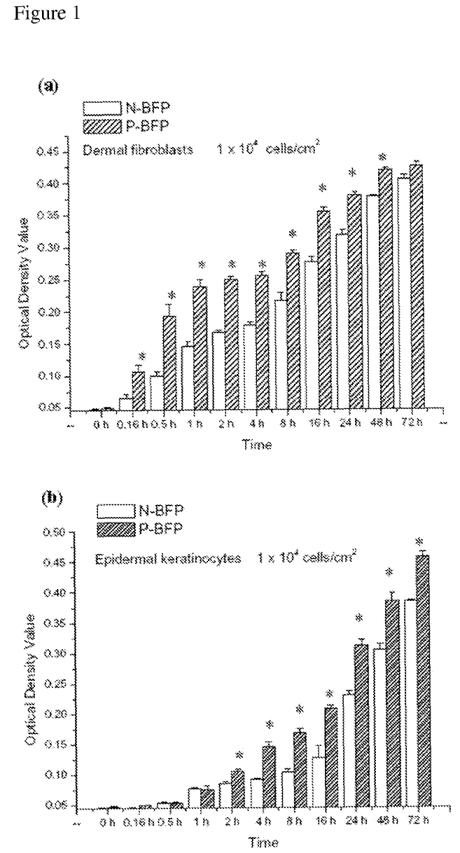

The construction of BFP was modified by Wierzba and Kilburn (Wierzba A, et al., Biotechnol Bioeng 1995; 47: 147-154). In brief, the cellobiohydrolase I gene (CBH I) has been cloned from an enhanced cellulase-producing strain, Trichoderma koningii. The GRGDS cell attachment sequence was then fused to the Trichoderma koningii CBH I gene CBD to serve as a linking molecule between the cell and the substrate. This GRGDS sequence was accommodated in a stable annular structure built up by disulfide bonds of cysteine and grafted on the N-terminal of CBD. Another GRGDS sequence was fused in a linear-free form and was grafted on the C-terminal of CBD (FIG. 1). In this way, there were two GRGDS sequences that could be recognized by the integrin receptors on the cell surface and enable the cells to attach onto the cellulose substrate firmly.

### Example 2

### Coating Procedure of BFP onto Petri Dish

BFP powder was dissolved in double-deionized water, with the pH value of 7.2. The prepared BFP solution was diluted to a concentration of 0.2 mg/mL and sent through a 0.22-m filter for sterilization. The coating plate was prepared by adding 0.1 mL of BFP solution into a 48-well plate and 0.25 mL of BFP solution into a 24-well plate, respectively. The final coating density was 0.02 mg/cm² in both groups. The plate was gently shaken for a while and placed for 30 min at room temperature for BFP grafting. Finally, the residual liquid was removed, and the plate was dried by laminar flow.

### Example 3

### Primary Culture of Keratinocytes and DFs

Fresh human adult foreskin biopsies were washed using phosphate buffered saline, with 1% penicillin-streptomycin-gentamycin antibiotics (Gibco Invitrogen, Carlsbad, Calif.). The harvested foreskins were then minced into small fragments. The fragments were immersed in 40 U/mg thermolysin as received (Sigma, St. Louis, Mo.) at 4° C., overnight. For isolation of keratinocytes, the epidermis was separated from the dermis with forceps, and then incubated in 0.1% EDTA solution with 0.05% trypsin addition (Gibco Invitrogen) for 15 min at 37° C. The isolated keratinocytes were cultivated and expanded by commercialized keratinocyte serum-free culture medium (Gibco Invitrogen), with medium supplement addition (epidermal growth factor, bovine pituitary extract) in a final concentration of 5 ng/mL and 50 g/mL, respectively. The dermis was treated with 0.2% collagenase (Sigma) at 37° C. for 1 h to isolate DFs. The isolated DFs were cultured in DMEM (Dulbecco's modified Eagle's medium) supplemented with 10% fetal bovine serum at 37° C., in 5% CO₂.

### Example 4

### Colorimetric MTT Assay for Cell Viability

The mitochondrial activity of the cultured cells was determined by calorimetric assay, which would be in terms of cell viability. In general, mitochondrial dehydrogenases detected the conversion of 3-(4,5-dimethylthiazolyl-2)-2,5-diphenyltetrazolium bromide (MTT, Sigma) to dark-blue formazan crystals.

The MTT assay was shortly described as follows: 1×10⁴ cells per well were cultured (5% CO₂, 37° C.) in 48-well petri dish. The experiment was divided into two groups. One group was grafted BFP (P—BFP) and the other one was nongrafted (N—BFP). At each experimental time period, the cultured medium was removed and 100 L of MTT solution (1 mg/mL in test medium) was added to the culture well. All wells were then further incubated for 4 h to allow the conversion of MTT to formazan crystals. The cultured medium was then removed, and acid isopropanol (100 L of 0.04N HCT in isopropanol) was added to all wells and mixed thoroughly to dissolve the dark-blue formazan crystals. After the crystal dissolved, the plates were read on a Micro Elisa reader (Emax Science, Sunnyvale, Calif.), with a test wavelength of 570 nm, against a reference wavelength of 690 nm. Plates were normally read within 1 h after isopropanol was added.

As shown in FIG. 1(a), the MTT assay of DFs at various cultured periods, with BFP coating group (P—BFP) and without BFP coating group (N—BFP). The optical density (OD) value of MTT assay, both in P—BFP and N—BFP group, increased throughout the culture periods. The DFs cellular density in the P—BFP group increased faster than that of N—BFP group from the initial seeding time point of 0.16 h and up to 24 h. The OD of MTT assay in the P—BFP group was higher than that of N—BFP group, ranging from 63% at 0.16 h to 19% at 24 h of culture time. The P—BFP group showed no significant difference in OD value from 48 to 72 h due to cell confluence.

As shown in FIG. 1(b), the MTT OD value of epidermal keratinocytes cultured at various time periods for the P—BFP and N—BFP group. It showed similar tendency to DFs, because the cell viability increased with the culture period in both groups. Nevertheless, the epidermal keratinocytes cultured in P—BFP and N—BFP group showed significant difference in each culture period, even at the end of 72 h.

### Example 5

### Total DNA Analysis for Quantification of Cell Growth

The purpose of total DNA analysis was to quantify the cell proliferation in 24-well petri dish in P—BFP and N—BFP group. The WizardR Genomic DNA Purification kit (Promega, Madison, USA) was chosen for DNA extraction. The first step was to lyse the cells and the nuclei with nuclei lysis solution, and it was then followed by an exhaustive RNase digestion step. The cellular proteins were then removed by salt-precipitation method, so that the high molecular weight genomic DNA was left in the solution. Finally, the genomic DNA was concentrated and desalted by isopropanol precipitation. The ratio of 260 and 280 nm absorption value on UV-vis spectrum should be in the range of 1.8-2.0 to yield a good deproteinization.

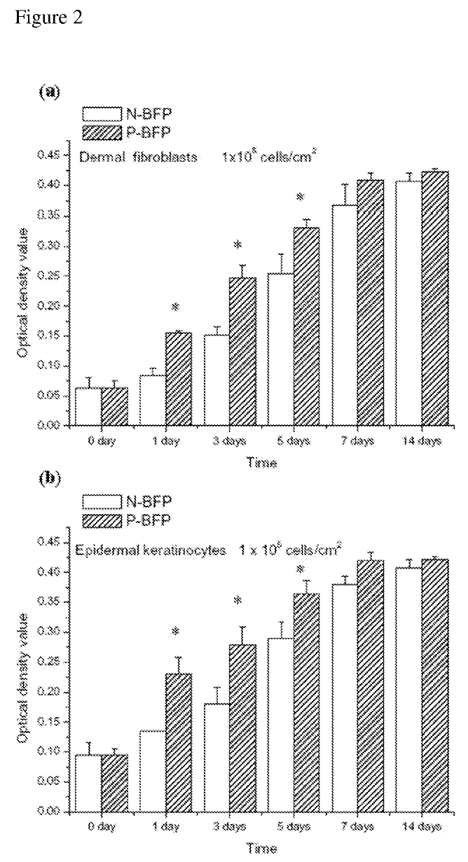

FIG. 2(a) showed the result of the genomic DNA quantification test. The OD value of P—BFP group in DFs showed no significant difference between the P—BFP and N—BFP group at 0 day. The OD value of total DNA in P—BFP group at day 1 and 3 were 0.155 and 0.248, respectively, and the OD value of total DNA in N—BFP group at day 1 and 3 were 0.084 and 0.152, respectively. At day 1 and 3, OD value of total DNA for two groups showed a significant difference (*p<0.05). The difference was 84% at the first day and 63% at the third day. The OD value of the total DNA for the P—BFP and N—BFP group showed no significant difference at day 7 and 14.

FIG. 2(b) showed the OD value of total DNA for P—BFP group and N—BFP group of cultured keratinocytes at different culture periods. At day 0, there was no obvious difference in OD value between the P—BFP and N—BFP group. At days 1, 3, and 5, the OD value of P—BFP group was 0.23, 0.28, and 0.365, respectively. The OD values of N—BFP group at days 1, 3, and 5 were 0.135, 0.18, and 0.29, respectively, which were much lower than that of P—BFP group, with 70% at day 1, 55% at day 3, and 26% at day 5. In addition, the OD values for fibroblasts and keratinocytes in P—BFP group had reached a plateau after culturing for about 7 days.

### Example 6

### Analysis of Lactate Dehydrogenase in the Medium for Cell Cytotoxicity

The lactate dehydrogenase (LDH) release from the lysed cells was measured by the commercialized assay kit (cat. MK401, Takara, Japan), which would be used to evaluate the cell damaged condition as so-called cytotoxicity. The method was described in detail as follows: 5×10³ cells/200 L per well were seeded on a 96-well plate. Following incubation for 1 day, the medium was refreshed by the combination solution of 100 L fresh medium and 100 L BFP solutions (in various concentrations). The plates were incubated for another 24 h. On the next day, the plates were used for LDH analysis. In brief, the plates were centrifuged for 250 g for 10 min at room temperature. From all wells, 100 L was transferred to a new plate, and 100 L LDH substrate solution was subsequently added. The LDH was detected by a two-step reaction. First, LDH catalyzed the conversion of metabolized lactate to pyruvate in the presence of NAD+. Second, the formed NADH reduced the tetrazolium salt to a red formozan. The increase in absorption of the chromogen during 15 min was determined at 490 nm on a spectrophotometer. Subsequently, the percentage of LDH leakage, relative to the control, was calculated in each concentration according to the equation as follows:

Cytotoxicity(%)=(exp. value−low control/high control—low control)×100

In this equation, high control was the maximum absorption of cells incubated with 1% Triton X-100; low control was the mean spontaneous absorption of cells incubated with medium; and experimental value was the absorption of cells incubated with different concentration of BFP solutions (0.01, 0.02, 0.1, and 0.2 mg/mL).

The LDH assay was an indirect method to evaluate the cell damage condition because of the LDH release from the mitochondria of lysed cells. LDH level in the culture medium would be measured with commercialized assay kit by ELISA. The result could be used for cytotoxic evaluation.

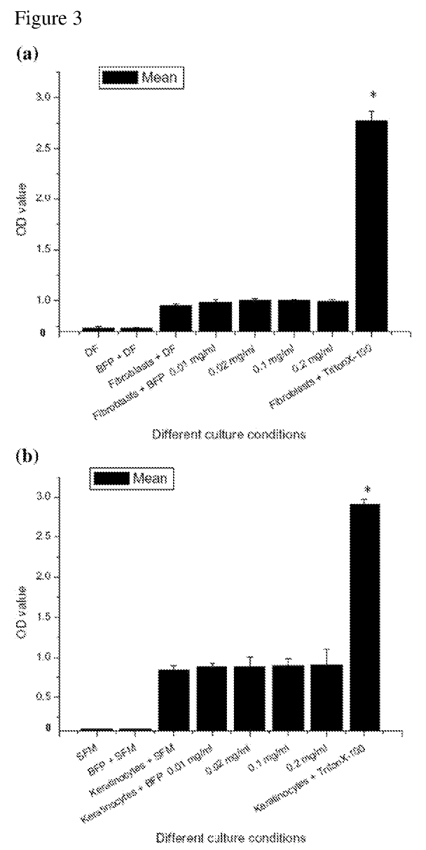

FIG. 3(a) showed the results of LDH test on DFs. In FIG. 3(a), the samples were classified into eight groups, with the abbreviation and meaning as follows: (1) DMEM medium+10% FBS without any DF; (2) 0.2 mg/mL of BFP added in DF medium without any DF (DF+BFP); (3) DF medium cultured with DFs without BFP added (fibroblasts+DF); (4) 0.01 mg/mL of BFP added in DF medium and cultured with DFs (fibroblasts+BFP 0.01 mg/mL); (5) 0.02 mg/mL of BFP added in DF medium and cultured with DFs (fibroblasts+BFP 0.02 mg/mL); (6) 0.1 mg/mL of BFP added in DF medium and cultured with DFs (fibroblasts+BFP 0.1 mg/mL); (7) 0.2 mg/mL of BFP added in DF medium and cultured with DFs (fibroblasts+BFP 0.2 mg/mL); (8) DFs cultured in DF with TritonX-100 addition used as the positive control because TritonX-100 is highly toxic to all kinds of cells (fibroblasts+TritonX-100).

DF group and DF+BFP group were used as the negative control because of no cell involvement, and no LDH release was supposed. The OD value for the two groups was close to ground value. The OD value of LDH for the fibroblasts+TritonX-100 group was much higher than the other groups because of positive control. Under these concentrations (0.01, 0.02, 0.1, and 0.2 mg/mL), BFP did not induce significant LDH release (<3%). The OD value of the LDH test showed no significant difference for all the test groups of DFs cultured with DF by different concentrations of BFP addition. Data also showed no significant difference with the group of fibroblasts+DE

FIG. 3(b) showed the results of LDH test on keratinocytes. In FIG. 3(b), the samples were also classified into eight groups with the abbreviation and meaning as follows: (1) serum-free medium (SFM) without any keratinocyte as one of negative control; (2) 0.2 mg/mL of BFP added in SFM without any keratinocyte (SFM+BFP) as another one negative control; (3) SFM cultured with keratinocytes without BFP added (keratinocytes+SFM); (4) 0.01 mg/mL of BFP added in SFM and cultured with keratinocytes (keratinocytes+BFP 0.01 mg/mL); (5) 0.02 mg/mL of BFP added in SFM and cultured with keratinocytes (keratinocytes+BFP 0.02 mg/mL); (6) 0.1 mg/mL of BFP added in SFM and cultured with keratinocytes (keratinocytes+BFP 0.1 mg/mL); (7) 0.2 mg/mL of BFP added in SFM and cultured with keratinocytes (keratinocytes+BFP 0.2 mg/mL); (8) keratinocytes culture in SFM with TritonX-100 addition as the positive control (keratinocytes+TritonX-100).

The OD value for the SFM group and SFM+BFP group was close to ground value because of negative control. The OD value of LDH for the keratinocytes+TritonX-100 group was much higher than those of the other groups because of positive control. The cytotoxicity was evaluated in the range from 1.83 to 2.83%. The OD value of the LDH test showed no significant difference for all the test groups of keratinocytes cultured with SFM by different concentrations of BFP addition. Data also showed no significant difference with the group of kertinocytes+SFM.

### Example 7

### Scanning Electron Microscope for Cell Morphology

For the scanning electron microscopic (SEM) examination, cells seeded on the BFP coating group (P—BFP) and without coating group (N—BFP) were both fixed in 4% paraformaldehyde, buffered with 0.1 M phosphate solution, for 2 h. Next, the samples were dried with serial increasing graded alcohols followed by critical-point drier processing. The surface of the wells was coated with ultrathin layer of gold/Pt in an ion sputter PVD chamber. The morphology and the protruded filopodia of cultured cells were evaluated with different time intervals. Keratinocytes in adhesion to BFP coated or uncoated substrate were observed by SEM. It is suggested that SEM could provide more informative message of the overall morphology of the interaction between the cells and the matrices than those observed under optical microscope.

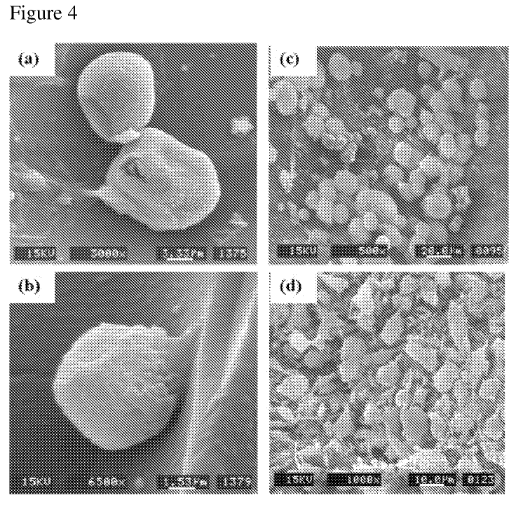

As shown in FIG. 4(a), the keratinocytes were seeded on the BFP uncoated petri dish for 2 h. The cells exhibited a round shape and lack of filopodia stretching. In contrast, the kertinocytes, well attached on the BFP coated petri dish with pseudopodia leading edge, extended out to the BFP substrate after seeding for 2 h.

After culturing on BFP uncoated petri dish for 8 h, most of the keratinocytes remained round in shape, and only a few cells stretched out and firmly anchored on the substrate, as shown in FIG. 4(c). In comparison with keratinocytes cultured on the BFP coated petri dish for 8 h, it demonstrated from FIG. 4(d) that more keratinocytes attached on the BFP coated petri dish and much more keratinocytes stretched out filaments to anchor on or spread on the coated petri dish. These evidences visualized the effect of bifunctional fusion protein in promoting keratinocytes attachment. After culturing on BFP uncoated petri dish for 8 h, most of the keratinocytes remained round in shape, and only a few cells stretched out and firmly anchored on the substrate, as shown in FIG. 4(c).

### Example 8

### Immunocytochemistry Analysis for Cell Phenotype

The monoclonal anti-pan cytokeratin antibody recognized human cytokeratins 4, 5, 6, 8, 10, 13, and 18 in immunohistochemistry. It was a broad-spectrum antibody, which reacted specifically with a wide variety of normal, reactive, and neoplastic epithelial tissues. In brief, samples were fixed with 4% formaldehyde for 2 h. Blocking was conducted with 10% goat serum to prevent unspecific binding. Specimens were washed with PBS for several times. Monoclonal anti-pan cytokeratin primary antibody (dilution 1:200; cat: c2931; Sigma) was incubated with the sample for 1 h at 37° C.

Next, anti-mouse IgG horseradish peroxidase (HRP) secondary antibody (Sigma) was then incubated with the samples for 30 min after the specimens were washed with PBS. SIGMA FASTR 3,3-diaminobenzidine tablets (DAB buffered tablets, urea hydrogen peroxide, Tris buffer) were used for staining.

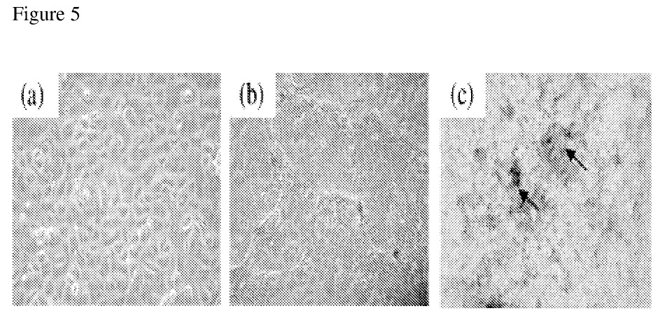

Monoclonal anti-pan cytokeratin was used to identify whether keratinocytes cultured on BFP coated petri dish kept their phenotype. After culturing for 3 days and then examining under an inverted optical microscope, it demonstrated from FIG. 5(a) that keratinocytes appeared as horn in shape that did not lose its original morphology.

As shown in FIG. 5(b), the keratinocytes cultured on BFP coated petri dish for 14 days and stained without monoclonal anti-pan cytokeratin first antibody treatment, as negative control, which exhibited no brown color expression. As shown in FIG. 5(c), the keratinocytes cultured on BFP coated petri dish for 14 days and treated with monoclonal anti-pan cytokeratin stain. There was dark brown in the middle of the culture area, and the keratinocytes differentiated into characteristic stratified epithelial layers. This demonstrated that there was no influence of bifunctional fusion protein on keratinocytes cell activity, and keratinocytes could normally secrete their own specific keratin protein under in vitro culture period.

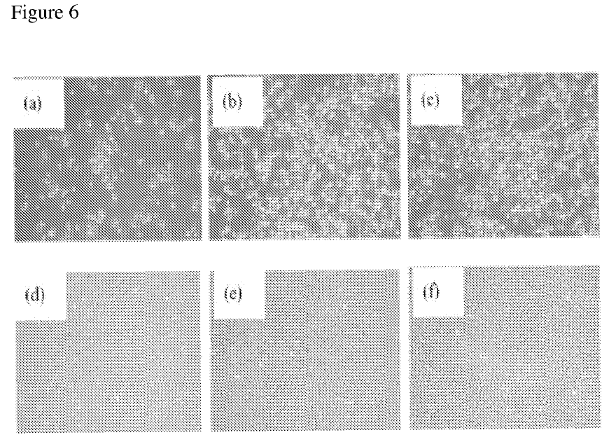

FIG. 6 demonstrated the morphological comparison of keratinocytes and DFs cultured in three different conditions: BFP uncoated plate as shown in FIGS. 6(a) and 6(d); RGD graft plate as shown in FIGS. 6(b) and 6(e); and BFP coated plate as shown in FIG. 6(c) and 6(f). Cell numbers of keratinocytes and DFs in BFP coated plate were much more than either BFP uncoated or RGD graft plate on day 3. In addition, keratinocytes expressed cobble-stone shape and DFs showed spindle shape in the three culture conditions. This suggested that both keratinocytes and DFs kept their phenotype without losing their cell viability.

All data in the examples were expressed as mean standard deviation, and were analyzed by analysis of variance (one-way ANOVA). Statistical significance was determined by Bonferroni's t-test. Probability values less than 0.05 were considered significant.

One skilled in the art readily appreciates that the present invention is well adapted to carry out the objects and obtain the ends and advantages mentioned, as well as those inherent therein. The processes and methods for measuring antibiotic resistant organism are representative of preferred embodiments, are exemplary, and are not intended as limitations on the scope of the invention. Modifications therein and other uses will occur to those skilled in the art. These modifications are encompassed within the spirit of the invention and are defined by the scope of the claims.

It will be readily apparent to a person skilled in the art that varying substitutions and modifications may be made to the invention disclosed herein without departing from the scope and spirit of the invention.

All patents and publications mentioned in the specification are indicative of the levels of those of ordinary skill in the art to which the invention pertains. All patents and publications are herein incorporated by reference to the same extent as if each individual publication was specifically and individually indicated to be incorporated by reference.

The invention illustratively described herein suitably may be practiced in the absence of any element or elements, limitation or limitations, which are not specifically disclosed herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

What is claimed is:

1. A method for improving cell-attachment efficiency comprising the steps of:
(a) preparing a protein in aqueous solution, wherein the protein has a formula A-B-C
wherein
A represents a GRGDS amino acid sequence;
B represents a cellulose binding domain (CBD); and
C represents a GRGDS amino acid sequence, an RGD amino acid sequence, or an amino acid sequence of growth factor;
(b) coating the protein solution into a carrier; and
(c) seeding cells onto the carrier.

2. The method as claimed in claim 1, which further comprises a step of shaking the carrier for fixation of the protein between step (b) and step (c).

3. The method as claimed in claim 2, which further comprises a step of drying the carrier to remove residual liquid between shaking step and step (c).

4. The method as claimed in claim 1, wherein the CBD is isolated from Trichoderma koningii CBH I gene.

5. The method as claimed in claim 1, wherein the protein further comprises a proline and threonine-rich linker (PT linker) between A and B or B and C.

6. The method as claimed in claim 1, wherein the GRGDS sequence expresses in a stable annular structure or in a linear-free form.

7. The method as claimed in claim 6, wherein the annular structure is built by disulfide bonds of cysteine residues.

8. The method as claimed in claim 1, wherein the cell is an alive cell.

9. The method as claimed in claim 1, wherein the cell is a skin cell, a stem cell or a delayed-action attachment cell.

10. The method as claimed in claim 9, wherein the skin cells is human epidermal keratinocytes or dermal fibroblasts.

11. The method as claimed in claim 1, which is carried out in aqueous solution without losing the 3D conformation of bio-molecules.

12. The method as claimed in claim 1, which is carried out in the absence of any organic solvent or crosslinking agent.

13. The method as claimed in claim 1, which maintains viability, proliferation, morphology, cytotoxicity, differentiation, migration and phenotype of the cell.

14. The method as claimed in claim 1, which can be applied to cosmetics for improving skin health.

15. A protein for improving cell-attachment efficiency which has a formula A-B-C
wherein
A represents a GRGDS amino acid sequence;
B represents a cellulose binding domain (CBD); and
C represents a GRGDS amino acid sequence, an RGD amino acid sequence, or an amino acid sequence of growth factor.

16. The protein as claimed in claim 15, wherein the CBD is isolated from Trichoderma koningii CBH I gene.

17. The protein as claimed in claim 15, which further comprises a proline and threonine-rich linker (PT linker) between A and B or B and C.

18. The protein as claimed in claim 15, wherein the GRGDS sequence expresses in a stable annular structure or in a linear-free form.

19. The protein as claimed in claim 18, wherein the annular structure is built by disulfide bonds of cysteine residues.

20. The protein as claimed in claim 15, which can be applied to cosmetics for improving skin health.


SEQUENCE LISTING (3 sequences)

SEQ ID NO: 1 - DNA, 591 bp, Trichoderma koningii
  CDS (1)..(591)
atgagcgataaaattattcacctgactgacgacagttttgacacggatgtactcaaagcggacggggcgatcctcgtcgatttctgggcagagtggtgcggtccgtgcaaaatgatcgccccgattctggatgaaatcgctgacgaatatcagggcaaactgaccgttgcaaaactgaacatcgatcaaaaccctggcactgcgccgaaatatggcatccgtggtatcccgactctgctgctgttcaaaaacggtgaagtggcggcaaccaaagtgggtgcactgtctaaaggtcagttgaaagagttcctcgacgctaacctggccggttctggttctggccatatgcaccatcatcatcatcattcttctggtctggtgccacgcggttctgccatggcatgcggtcgtggtgacagctgtccaactactccaaccatggctacccagtctcactggggccagtgcggcggtattggctacagcggccccacggtctgcgccagcggcacaacttgccaggtcctgaacccttactactctcagtgcctgccaactactccaactggtcgtggtgacagcgctagctga

SEQ ID NO: 2 - protein, 196 aa, Trichoderma koningii
MSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLAGSGSGHMHHHHHHSSGLVPRGSAMACGRGDSCPTTPTMATQSHWGQCGGIGYSGPTVCASGTTCQVLNPYYSQCLPTTPTGRGDSAS

SEQ ID NO: 3 - protein, 196 aa, Trichoderma koningii
  PEPTIDE (1)..(196)
MSDKIIHLTDDSFDTDVLKADGAILVDFWAEWCGPCKMIAPILDEIADEYQGKLTVAKLNMDQNPGTAPKYGIRGIPTLLLFKNGEVAATKVGALSKGQLKEFLDANLAGSGSGHMHHHHHHSSGLVPRGSAMACGRGDSCPTTPTMATQCHWGQCGGIGYSGPTVCASGTTCQVLNPTTSQCLPTTPTGRGDSAS